(19) United States
(12) Patent Application Publication
(10) Pub. No.: US 2004/0110678 A1
(43) Pub. Date: Jun. 10, 2004

(54) Novel drug delivery system

(76) Inventors: Giuliano Siligardi, Surrey (GB); Rohanah Hussain, Surrey (GB)

Correspondence Address: DeWitt Ross & Stevens, Intellectual Property Department, US Bank Building Suite 401, 8000 Excelsior Drive, Madison, WI, 53717-1914, US

(21) Appl. No.: 10/468,112

(22) PCT Filed: Feb. 15, 2002
(86) PCT No.: PCT/GB02/00680

(30) Foreign Application Priority Data
Feb. 16, 2001 (GB) 01038777

(51) Int. Cl. A61K 38/38

(52) U.S. Cl. 514/12; 530/363

(57) ABSTRACT

An exogenous pharmaceutical preparation in which a lipid tagged bioactive material is combined with albumin is disclosed. The albumin has a low proportion of lipidic/fatty acid groups (a molar ratio of fatty acid groups to albumin of less than 0.7) and is preferably fatty acid free. The albumin may be naturally extracted such as Human Serum Albumin or recombinantly produced. A range of bioactive substances, such as peptides, proteins or vaccines may be tagged and combined with albumin for human, veterinary or agricultural use. Several antimicrobial peptides are disclosed. Binding to the albumin mediates the biostability of the tagged bioactive material. Due to this stabilization, the antimicrobial activity of a model lipopeptide is enhanced when combined with substantially fatty acid free-albumin. When bound to albumin, therefore, the model lipopeptide exerts an antimicrobial effect at a lower concentration than the model lipopeptide alone.

This invention relates to systems for the delivery of bioactive materials.

The search for effective drug delivery systems is still a major problem of pharmaceutical research, especially for biomedically important molecules such as proteins, peptide vaccines, peptide-base tumour therapeutic agents, and peptide-base antimicrobials. The use of lipidic moieties covalently attached to these drugs has been proposed to improve absorption and transportation in vivo. However this strategy has met with limited success. Biostability to enzymatic degradation still poses a problem with the peptide-base drugs containing lipidic moieties. To counteract this problem an increased number of lipidic moieties have to be covalently-linked to these drugs which poses another formulation problem i.e. solubility for in vivo administration.

International patent application) WO 92/01476 is directed to the covalent attachment (tagging) of a fatty acid group to a protein or peptide drug. The purpose of tagging the drug in this manner is that the tagged drug when administered will attach itself non-covalently to albumin circulating in vivo. WO 92/01476 discusses the availability of binding sites on the albumin molecule and the fact that fatty acids can bind at these sites through hydrophobic interactions. It is also stated that in plasma there are still vacant binding sites in the albumin molecule even though fatty acids have previously bound at other sites. The presence of naturally bound fatty acid in the albumin molecule is assessed in various ways in the published literature, ranging from up to 1 or 2 fatty acid molecules per molecule of albumin (increasing to 4 during strenuous exercise). WO 92/01476 asserts that the tagged drug can be administered as it stands and that it will bind rapidly to endogenou's albumin. It also contemplates co-administration of albumin and tagged drug as separate entities.

We have now found that tagged bioactive materials may be used successfully by combining the tagged material with albumin in the form of an exogenous preparation, providing that the albumin used initially contains much lower proportions of fatty acid than has been previously contemplated for endogenous preparations, namely no more than 0.5 moles fatty acid per mole or albumin. For best results, the albumin used is substantially free from fatty acid molecules, allowing the fatty acid tagged drug to take up most or all of the binding sites available in the fatty acid-free albumin. Relatively fat-free albumin can be obtained commercially or can be prepared to any desired fatty acid by the use of known methods.

When using human serum albumin all the albumin of choice, and in the fatty acid-free form (HSAff), we have found that the HSAff enhances the antimicrobial activity of the model lipopeptide used. With this model lipopeptide, the effect is twice as potent as compared to the lipopeptide alone or with human serum albumin containing already bound fatty acid (HSAfa). The difference in potency widens more significantly when samples are subjected to appropriate enzymatic degradative conditions. This is consistent with the protective effect of HSAff on the lipopeptide towards digestive enzymes in the body. The results demonstrate that fatty acid free albumin (HSAff) can be used as external ingredient to enhance the biological activities of other lipo drugs by mediating their biostability.

The present invention comprises an exogenous pharmaceutical preparation comprising a bioactive substance covalently attached to a lipidic (fatty acid) tagging group, the tagged substance being non-covalently bound to an albumin initially containing a low proportion of lipidic groups as indicated above. Preferably, the lipidic tagging group is a C₄-C₁₆ single chain fatty acid. The exogenous pharmaceutical preparations according to the present invention preferably make use of albumin which is initially substantially fatty acid-free.

U.S. Pat. No. 4,094,965 is directed to diagnostic compositions and describes a method for preparing a clear solution of radiolabelled albumin which is stable over a wide pH range. Whereas solutions of standard albumin containing stannous ions tend to be cloudy, it is stated that fat free human serum albumin, HSA, is more stable and forms clear solution in a mixture containing reducing agent and radionuclide.

The present invention is applicable to the formulation of many types of bioactive material including

Drugs or molecules naturally or synthetically produced containing lipo/lipid moieties.

Peptides containing lipidic moiety either natural or synthetic.

Peptidomimetics containing lipidic moiety either natural or synthetic.

Protein containing lipidic moiety either natural or synthetic.

Glycopeptides containing lipidic moiety either natural or synthetic.

Natural or synthetic lipopeptides as vaccines (Steller et al. Cancer Res 1996, 5087-91; von Herrath et al. Virology 2000, 268, 411-9; Loing et al. J. Immunol 2000, 164, 900-7; Wiesmuller et al. Biol Chem 2001, 382, 571-9).

All types of albumin may be used including naturally extracted or recombinantly produced albumins.

Determination of Fatty Acid Contents in Albumin

The amount of fatty acids in HSAff and HSAfa was ascertained by the displacement of bound diazepam at the fatty acid binding site of albumin and determined by circular dichroism (CD) spectroscopy. Induced CD was observed, which is indicative of detectable interaction between diazepam and HSA. This is consistent with the fatty acid preventing any diazepam binding to albumin.

Mutual Stabilisation Effect

As a preliminary to drug formulation, we have ascertained the mutual stabilisation effect of HSAff and HSAfa with model lipopeptides GS01, RH01, Tric 1.8 and Tric 4.8 in the presence of Pronase® (a mixture of exo- and endo-peptidases) 1/100 w/w to HSA in vitro.

With these lipopeptide models, the tagged peptides bind to the fatty acid binding site of HSAff as monitored by the displacement of diazepam, thus protecting the HSAff from hydrolysis. This effect gave rise to enhanced stability of HSAff to proteolysis. When these peptides were used in conjunction with HSAfa, no further enhancement of stability to proteolysis was observed. The lipopeptides stabilise HSAff and in doing so they themselves are stabilised by HSAff resulting in mutual stabilisation.

Minimum Inhibitory Concentration Determination

Also as a preliminary to drug formulation, we have ascertained the minimum inhibitory concentration (MIC) of the antimicrobial model lipopeptide RH01 in the presence of human serum albumin fatty acid free compared to that of the peptide in the presence of human serum albumin with fatty acid for Escherichia coli and Staphylococcus aureus bacteria.

With this lipopeptide model, the tagged peptide bind to the fatty acid binding site of HSAff, thus resisting hydrolysis by bacterial enzymes and is able to exert its antimicrobial activity at a lower concentration. When the peptide is used in conjunction with HSAfa, the MIC remains the same as that of the peptide alone indicating that HSAfa does not confer further stability to the peptide. Since most of the peptide is not bound to HSAfa, RHOL is susceptible to bacterial enzymatic degradation.

The difference in the MIC between samples containing HSAfa and HSAff was more pronounced, increased to four times, when incubated with a higher Pronase®, concentration of 1/250 w/w to the lipopeptide.

The MIC between the samples in the presence of varying concentrations of fatty acid were also adversely affected.

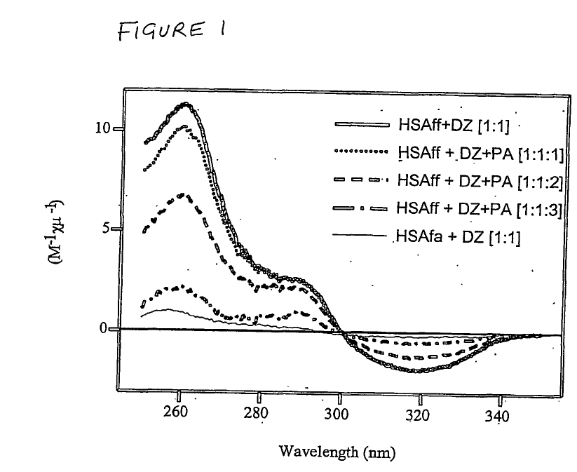

FIG. 1 shows the displacement of bound diazepam (DZ) to albumins (HSAff and HSAfa) by palmitic acid (PA);

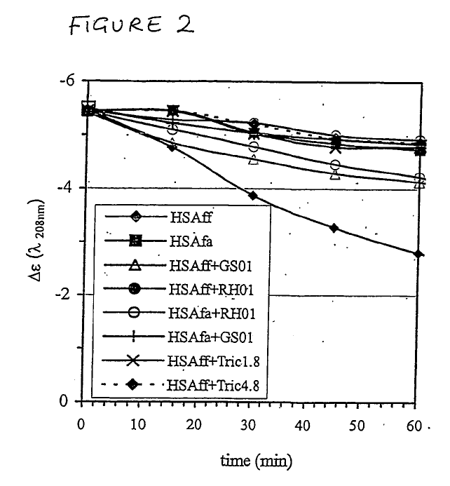

FIG. 2 demonstrates the effect of lipopeptides GS01, RH01, Tric 1.8 and Tric 4.8 on albumins (HSAff and HSAfa) incubated with Pronase® (1/100 w/w) degradation conditions.

### EXAMPLES

RH01 is a myristoylated nonapeptide (1) and GS01 is a myristoylated undecapeptide (2). RH01, was synthesised by peptide solid phase synthesis (see example 28) and GS01 was purchased from Advanced Biotech Centre, Imperial College, London. Tric 1.8 (3) and Tric 4.8 (4) were octylated undecapeptides synthesised by solution phase as reported by Monaco, Formaggio, et al. and Milhauser, Biopolymers, 1999, 50, 239.

CH₃(CH₂)₁₂CO—X where X

(1) FARKGALRQ and

(2) FQWQRNMRKVR

CH₃(CH₂)₆CO—X where X is

(3) Toac-GL-Aib-GGL-Toac-GIL(Me) and

(4) Aib-GL-Toac-GGL-Toac-GIL(Me),

Toac is (2,2,6,6-tetra-methypiperidine-1-oxyl-4-amino-4-carboxylic acid)

Aib is (2-aminoisobutyric acid)

The results of the following investigations (Examples 1-3) follow after example 3.

### Example 1

### Content of Fatty Acids in Albumin Fatty Acid Free Analysed by Diazepam Displacement

Essentially fatty acid free (approx 0.005% that corresponds to 0.0002M/M albumin) and essentially globulin-free human albumin, HSAff, (lot. 32H9300) was purchased from Sigma-Aldrich Company Ltd (Fancy Road, Poole, Dorset, BH12 4QH, England).

HSAff (1.834 mg) was dissolved in distilled water (3.057 ml). Diazepam (0.088 mg) was dissolved by sonication for 30 mins in distilled water (0.687 ml). Sodium palmitate (0.872 mg) was dissolved in water pH 8.8 (0.698 ml) by sonicating for 5 mins. HSAff solution (2.550 ml) was transferred to 5 cm cell and CD spectrum was recorded. Diazepam (51 μl) was added to the HSAff in the 5 cm cell and the mixture was gently mixed by rotating the cell several times. The CD spectrum of the mixture was then recorded. Sodium palmitate (51 μl) was added to the cell and the mixture was gently mixed by rotating the cell several times. The CD spectrum of the mixture was recorded. This process was repeated twice over, each time the CD spectrum was recorded. The cell was then washed thoroughly with distilled water and ethanol. Distilled water (2.705 ml) was then placed in the 5 cm cell and CD spectrum recorded.

CD spectra were recorded with nitrogen flushed JASCO spectropolarimeter J600 using 4 s time constant, 10 nm/min scan speed and a spectral bandwidth of 2 nm. The induced CD spectra of bound diazepam were obtained by subtracting the spectrum of albumin from that of the HSA-diazepam mixture. The spectra were reported as Δε=ε_{L}−ε_{R}(M⁻¹cm⁻¹).

### Example 2

### Content of Fatty Acids Analysed by Diazepam Displacement of Albumin Globulin-Free

Human serum albumin essentially globulin-free (HSAfa) was purchased from Sigma-Aldrich Company Ltd (lot. 105H9300).

HSAfa (1.857 mg) was dissolved in distilled water (3.095 ml). Diazepam (0.088 mg) was dissolved by sonication for 30 mins in distilled water (0.687 ml). HSAfa solution (2.705 ml) was transferred to 5 cm cell and CD spectra was recorded. Diazepam (55 μl) was added to the HSAfa in the 5 cm cell and the mixture was gently mixed by rotating the cell several times. The CD spectrum of the mixture was then recorded. The cell was then washed thoroughly with distilled water and ethanol. Distilled water (2.705 ml) was then placed in the 5 cm cell and CD spectrum recorded. CD parameters are as in example 1.

### Example 3

### Diazepam Binding to Fatty Acid Free Bovine Serum Albumin

Bovine serum albumin essentially fatty acid free (approx 0.005%, corresponding to 0.0002M/M albumin) and essentially globulin-free. (BSAff) was purchased from Sigma-Aldrich Company Ltd (lot 100K7415).

BSAff (1.914 mg) was dissolved in distilled water (3.19 ml) to give concentration of 0.6 mg/ml. Diazepam (0.422 mg) was dissolved by sonication for 30 mins in distilled water (3.297 ml) to give a concentration of 0.128 mg/ml. BSAff solution (1.05 ml) was transferred to 2 cm cell and CD spectra was recorded. Diazepam (22 μl) was added to the BSAff in the 2 cm cell and the mixture was gently mixed by rotating the cell several times. The CD spectrum of the mixture was then recorded. The cell was then washed thoroughly with distilled water and ethanol. Distilled water (1.10 ml) was then placed in the 2 cm cell and CD spectrum recorded. CD parameters are as in example 1.

Results

Diazepam and medium chain fatty acids are known to bind to albumin site II as discussed by T Peters, All about albumin, Academic Press, 1999, p116. The fatty acid molecules are devoid of any CD signal. On the contrary diazepam shows a CD signal only when is bound to the albumin. In here, diazepam is used as a marker to show the binding of fatty acids and fatty acid containing molecules to albumin. In FIG. 1, the induced CD spectrum of diazepam shows the highest intensity when bound to fatty acid free HSAff than albumin with fatty acid HSAfa. The value of Δε intensity at 260 nm is 11.3 for HSAff and 0.9 for HSAfa, which correspond to 100% and 8% (0.9/11.3*100) respectively of percentage of diazepam induced CD. Palmitic acid binds to at least five long chain fatty acid binding sites of which one is closely located to albumin site II as reported by Curry et al. (1998, Nature Structural Biology, 5, 827-835). The percentage of diazepam induced CD for 1, 2 and 3 molar equivalents of palmitic acid added to the mixture HSAff-diazepam (1:1) is 88.5%, 60.0% and 18.6% respectively. The displacement of albumin-bound diazepam by palmitic acid indicates that palmitic acid affects the affinity of ligands in site II. As shown in FIG. 1, after addition of more than three molar equivalent of palmitic acid per molar HSAff (fatty acid >3M/M HSAff), the diazepam was almost displaced from the binding site (FIG. 1). The fact that the induced CD of diazepam bound to HSAff with sodium caprylate 5.4M/M (data not shown) is also identical to that of diazepam bound to HSAfa (FIG. 1) is consistent with sodium caprylate added to HSAfa in pasteurization at 5.4M/M (Peters, All about Albumin, Academic Press, 1996, p302). In the case of fatty acid containing molecules, in particular lipopeptide RH01, the diazepam marker test is applied to demonstrate the formation of fatty acid free HSAff-RH01 complex. The displacement of diazepam (DZ) illustrated by its decreased induced CD upon addition of RH01 at different molar ratios (data not shown) to the mixture HSAff-DZ (1:1) demonstrates the drug carrier property of fat free HSA.

The diazepam marker test is applied to Baxter HSA (Baxter Healthcare Ltd, Hyland Immuno, Wallingford Road, Compton, Newbury, Berks RG20 7QW) and delipidised Baxter HSA to ascertain their fatty acid (caprylic acid) content. The percentage of diazepam induced CD is 8% for Baxter HSA (fatty acid 5.4M/M) and 100% for delipidised Baxter HSA (data not shown). The induced CD of diazepam bound to delipidised Bax HSA is identical to that of diazepam bound to Sigma HSAff, which indicates that the content of fatty acid of delipidised Bax HSA has to be no more than 0.0002M/M as for HSAff.

Diazepam binding to recombinant fatty acid free HSA has been ascertained showing similar induced CD to that seen in FIG. 1 for HSAff-DZ (1:1).

Diazepam is also shown to bind to BSAff (fatty acid 0M/M) showing the characteristic induced CD as seen in FIG. 1 for human albumin. This is indicative of BSAff fatty acid binding property similar to that of HSAff which demonstrate the drug carrier property of fat free BSA.

The results of the following examples 4-14 are presented after example 14.

### Example 4

### Stability Studies of Fat Free Albumin in the Presence of Pronase® by Circular Dichroism

HSA fatty acid free (2.359 mg) was dissolved in distilled water (3.93 ml) to give concentration 0.60 mg/ml. Pronase® (0.363 mg) was dissolved in distilled water (0.605 ml) to give a concentration 0.60 mg/ml.

HSA fatty acid free solution (0.2 ml) was placed in a 0.05 cm cell and the circular dichroism spectrum (CD) was recorded. The cell was then thoroughly washed with distilled water and ethanol. HSA fatty acid free solution (2.7 ml) was placed in a glass vial. Pronase® solution (27 μl) was added to HSA fatty acid free solution in the glass vial. The mixture was mixed gently and 200 μl was transferred to 0.05 cm cell. The cell was then incubated at 37° C. and CD spectra recorded at various incubation time intervals.

CD spectra were recorded with nitrogen flushed JASCO spectropolarimeters J600 4s time constant, 10 nm/min scan speed and a spectral bandwidth of 2 nm. 0.05 cm pathlength cell was used to obtain an optimal CD signal and UV absorptions at a scanning wavelength 190-260 nm. The spectra were reported as Δε=εL−εR (M⁻¹cm⁻¹) using an average amino acid molecular weight 113.

### Example 5

### Stability Studies of Albumin with Fatty Acid in the Presence of Pronase® by Circular Dichroism

HSA with fatty acid (0.888 mg) was dissolved in distilled water (1.48 ml) to give concentration 0.60 mg/ml. Pronase® (0.085 mg) was dissolved in distilled water (142 μl) to give a concentration 0.6 mg/ml.

Experimental procedure was repeated as in Example 4.

### Example 6

### Stability Studies of Fat Free Albumin with Myristoylated Undecapeptide in the Presence of Pronase® by Circular Dichroism

HSA fatty acid free (2.063 mg) was dissolved in Tris HCl 10 mM buffer (3.44 ml) to give concentration 0.60 mg/ml. Pronase® (0.212 mg) was dissolved in tris HCl 10 mM (0.353 ml) to give a concentration 0.60 mg/ml. GS01 (0.194 mg) was dissolved in tris HCl 10 mM (231 μl) to give concentration of 0.84 mg/ml.

HSA fatty acid free solution (275 μl) was placed in a glass vial. GS01 (11 μl) was added to the HSA fatty acid free in the vial. Mixture was gently mixed. Mixture (200 μl) was transferred into a 0.05 cm cell and CD spectrum was recorded. The mixture in the cell was transferred back to the vial. The mixture (250 μl) was then transferred to another vial and Pronase® solution (2.5 μl) was added to this mixture. The mixture was mixed gently and 200 μl was transferred to 0.05 cm cell. The cell was then incubated at 37° C. and CD spectra recorded at various incubation time intervals.

Experimental procedure was repeated as in Example 4.

### Example 7

### Stability Studies of Fat Free Albumin with Myristoylated Nonapeptide in the Presence of Pronase® by Circular Dichroism

HSA fatty acid free (2.063 mg) was dissolved in Tris HCl 10 mM (3.44 ml) to give concentration 0.60 mg/ml. Pronase® (0.212 mg) was dissolved in tris HCl 10 mM (0.353 ml) to give a concentration 0.6 mg/ml. RH01 (0.184 mg) was dissolved in tris HCl 10 mM (153 μl) to give concentration of 1.206 mg/ml.

HSA fatty acid free solution (275 μl) was placed in a glass vial. RH01 (5.5 μl) was added to the HSA fatty acid free in the vial. Mixture was gently mixed. Mixture (200 μl) was transferred into a 0.05 cm cell and CD spectrum was recorded. The mixture in the cell was transferred back to the vial. The mixture (250 μl) was then transferred to another vial and Pronase® solution (2.5 μl) was added to this mixture. The mixture was mixed gently and 200 μl was transferred to 0.05 cm cell. The cell was then incubated at 37° C. and CD spectra recorded at various incubation time intervals.

Experimental procedure was repeated as in Example 4.

### Example 8

### Stability Studies of Albumin with Fatty Acid with Myristoylated Undecapeptide in the Presence of Pronase® by Circular Dichroism

HSA with fatty acid (0.383 mg) was dissolved in Tris HCl 10 mM (638 μl) to give concentration 0.60 mg/ml. Pronase® (0.212 mg) was dissolved in tris HCl 10 mM (0.353 ml) to give a concentration 0.6 mg/ml. GS01 (0.194 mg) was dissolved in tris HCl 10 mM (231 μl) to give concentration of 0.84 mg/ml.

HSA with fatty acid solution (275 μl) was placed in a glass vial. GS01 (11 μl) was added to the HSA with fatty acid in the vial. Mixture was gently mixed. Mixture (200 μl) was transferred into a 0.05 cm cell and CD spectrum was recorded. The mixture in the cell was transferred back to the vial. The mixture (250 μl) was then transferred to another vial and Pronase® solution (2.5 μl) was added to this mixture. The mixture was mixed gently and 200 μl was transferred to 0.05 cm cell. The cell was then incubated at 37° C. and CD spectra recorded at various incubation time intervals.

Experimental procedure was repeated as in Example 4.

### Example 9

### Stability Studies of Albumin with Fatty Acid with Myristoylated Nonapeptide in the Presence of Pronase® by Circular Dichroism

HSA with fatty acid (0.383 mg) was dissolved in Tris HCl 10 mM (638 μl) to give concentration 0.60 mg/ml. Pronase® (0.212 mg) was dissolved in tris HCl 10 mM (0.353 ml) to give a concentration 0.6 mg/ml. RH01 (0.184 mg) was dissolved in tris HCl 10 mM (153 μl) to give concentration of 1.206 mg/ml.

HSA with fatty acid solution (275 μl) was placed in a glass vial. RH01 (5.5 μl) was added to the HSA with fatty acid in the vial. Mixture was gently mixed. Mixture (200 μl) was transferred into a 0.05 cm cell and CD spectrum was recorded. The mixture in the cell was transferred back to the vial. The mixture (250 μl) was then transferred to another vial and Pronase® solution (2.5 μl) was added to this mixture. The mixture was mixed gently and 200 μl was transferred to 0.05 cm cell. The cell was then incubated at 37° C. and CD spectra recorded at various incubation time intervals.

Experimental procedure was repeated as in Example 4.

### Example 10

### Stability Studies of Fat Free Albumin with Trio 4.8 in the Presence of Pronase® by Circular Dichroism

HSA fatty acid free (2.063 mg) was dissolved in water (3.44 ml) to give concentration 0.60 mg/ml. Pronase® (0.212 mg) was dissolved in water 10 mM (0.353 ml) to give a concentration 0.60 mg/ml. Tric 4.8 (0.201 mg) was dissolved in methanol (1 ml) to give concentration of 0.20 mg/ml.

HSA fatty acid free solution (300 μl) was placed in a glass vial. Tric 4.8 (18 μl) was added to the HSA fatty acid free in the vial. Mixture was gently mixed. Mixture (200 μl) was transferred into a 0.05 cm cell and CD spectrum was recorded. The mixture in the cell was transferred back to the vial. The mixture (200 μl) was then transferred to another vial and Pronase® solution (2 μl) was added to this mixture. The mixture was mixed gently and 180 μl was transferred to 0.05 cm cell. The cell was then incubated at 37° C. and CD spectra recorded at various incubation time intervals.

### Example 11

### Stability Studies of Fat Free Albumin with Tric 1.8.in the Presence of Pronase® by Circular Dichroism

HSA fatty acid free (2.063 mg) was dissolved in water (3.44 ml) to give concentration 0.60 mg/ml. Pronase® (0.212 mg) was dissolved in water 10 mM (0.353 ml) to give a concentration 0.60 mg/ml. Tric 1.8 (0.09 mg) was dissolved methanol (0.450 ml) to give concentration of 0.20 mg/ml.

HSA fatty acid free solution (300 μl) was placed in a glass vial. Tric 1.8 (18 μl) was added to the HSA fatty acid free in the vial. Mixture was gently mixed. Mixture (200 μl) was transferred into a 0.05 cm cell and CD spectrum was recorded. The mixture in the cell was transferred back to the vial. The mixture (200 μl) was then transferred to another vial and Pronase® solution (2 μl) was added to this mixture. The mixture was mixed gently and 180 μl was transferred to 0.05 cm cell. The cell was then incubated at 37° C. and CD spectra recorded at various incubation time intervals.

Experimental procedure was repeated as in Example 4.

### Example 12

### Stability Studies of Commercial Baxter Human Serum Albumin (Bax HSA) (Baxter Healthcare Ltd, Hyland Immuno, Wallingford Road, Compton, Newbury, Berks RG20 7QW) in the Presence of Pronase® by Circular Dichroism

Human Albumin Solution 4.5% B.P. Immuno (Bax HSA) for clinical use was purchased from Baxter (Batch 033100I p20263Z). Bax HSA contains sodium caprilate 3.6 millimoles/l that corresponds to caprylate 5.4M/M albumin and sodium acetyltryptophanate 3.6 millimoles/l.

Bax HSA (40 μl) was added to water (2.96 ml) to give concentration 0.60 mg/ml. Pronase®D (0.135 mg) was dissolved in water (215 μl) to give a concentration 0.60 mg/ml.

Bax HSA 0.60 mg/ml solution (275 μl) was placed in a glass vial and Pronase® solution (2.5 μl) was added to the vial. The mixture was mixed gently and 200 μl was transferred to 0.05 cm cell. The cell was then incubated at 37° C. and CD spectra recorded at various incubation time intervals.

Experimental procedure was repeated as in Example 4.

### Example 13

### Stability Studies of Delipidised (Fat Free) Commercial Baxter Human Serum Albumin (DBax HSA) 2.21% in the Presence of Pronase® by Circular Dichroism

Bax HSA 4.5% (20 ml) was dialysed in 0.9% NaCl (2000 ml) in a beaker. The 0.9% NaCl solution was changed 6 times over 24 h. The delipidised Baxter HSA was collected and concentration ascertained spectroscopically at 278 nm with HSAff 4.5% as the reference. Delipidised Baxter HSA concentration was calculated as 2.21%.

DBax HSA 2.21% (81 μl) added to water (2.9 ml) to give concentration 0.60 mg/ml. Pronase® (0.316 mg) was dissolved in water (527 μl) to give a concentration 0.60 mg/ml.

DBax HSA 0.60 mg/ml solution (1.2 ml) was placed in a 0.05 cm cell and Pronase® solution (12 μl) was added to this cell. The mixture was mixed gently by rotating the cell several time. The cell was then incubated at 37° C. and CD spectra recorded at various incubation time intervals.

Experimental procedure was repeated as in Example 4.

### Example 14

### Stability Studies of Delipidised (Fat Free) Commercial Baxter Human Serum Albumin (DBax HSA) 2.21% with Lipopeptide RH01 (1:2) in the Presence of Pronase® by Circular Dichroism

DBax HSA 2.21% (81 μl) added to water (2.9 ml) to give concentration 0.60 mg/ml. Pronase® (0.316 mg) was dissolved in water (527 μl) to give a concentration 0.60 mg/ml. RH01 (0.322 mg) was dissolved in water (161 μl) to give concentration of 2 mg/ml.

DBax HSA 0.60 mg/ml solution (0.3 ml) was placed in a glass vial. RHOL 2 mg/ml (3.5 μl) was added to the DBax HSA in the vial. Mixture was gently mixed. Mixture (250 μl) was transferred into a 0.05 cm cell and CD spectrum was recorded. The mixture in the cell was transferred back to the vial. The mixture (250 μl) was then transferred to another vial and Pronase® solution (2.5 μl) was added to this mixture. The mixture was mixed gently and 180 μl was transferred to 0.05 cm cell. The cell was then incubated at 37° C. and CD spectra recorded at various incubation time intervals.

Experimental procedure was repeated as in Example 4.

Results

Upon incubation with Pronase®, the overall decrease in intensity of the far UV CD spectrum of the albumin as a function of time is related to the degree of enzymatic degradation. This is illustrated in the degradation plot of the CD intensity at 208 nm versus the incubation time (FIG. 2). The CD spectra were recorded as a function of time every 15 minutes up 90 minutes with the last measurement being at either 120 or 150 minutes.

The stability towards enzymatic degradation is calculated dividing the Δε value at 60 min by the Δε value at time zero for each experiment.

For the mixtures HSAff+lipopeptides (Tric1.8 and Tric4.8) the stability is 88% that corresponds to 12% of enzymatic degradation.

For the mixtures HSAfa+lipopeptides (GS01 and RH01) the stability is 88% that corresponds to 12% of enzymatic degradation (100-88).

For HSAfa the stability is 87% that corresponds to 13% of enzymatic degradation. For the mixtures HSAff+lipopeptides (GS01 and RH01) the stability is 78% that corresponds to 22% of enzymatic degradation.

For HSAff the stability is 51% that corresponds to 49% of enzymatic degradation.

HSA with and without Fatty Acid in Water in the Presence of Pronase® 1/100 w/w.

Upon incubation with Pronase® as a function of time, a greater reduction of the overall intensity of the CD at 208 nm is observed for HSAff than HSAfa (FIG. 2). The stability towards Pronase®D degradation of HSAfa is 87% whilst that of HSAff is 51% in agreement with the findings that fatty acid molecules bound to albumin have a substantial stabilizing effect (T Peters, All about albumin, Academic Press, 1995, p249).

HSAfa and HSAff in the Presence of GS01 (1:2) and RH01 (1:2) in Tris HCl 10 mM Buffer and HSAff in the Presence of Tric 1.8 and Tric 4.8 in Water in the Presence of Pronase® 1/100 w/w.

Upon incubation with Pronase®, HSAfa mixed with GS01 and RH01 showed a similar degradation profile to that of HSAfa alone (FIG. 2). This observation indicates that the lipopeptides do not confer further significant stability to HSAfa.

Lipopeptides have been found to stabilise substantially HSAff from 51% to 78% with both GS01 and RH01 and 89% with both Tric1.8 and Tric4.8 and in doing so the lipopeptides themselves are stabilised by HSAff resulting in mutual stabilisation. This is consistent with the enhanced antimicrobial activity of the formulation of lipopeptide containing HSAff as shown below in Example 15.

Baxter HSA (Bax HSA) and Delipidised Mat free) Baxter HSA (DBax. HSA) in the Presence of Pronase® 1/100 w/w.

Bax HSA 4.5% contains 3.6 mmol/L of Sodium Acetyl Tryptophanate and 3.6 mmol/L of Sodium Caprylate as stabilisers. Upon incubation with Pronase® as a function of time, a greater reduction of the overall intensity of the CD was observed for fat free DBax HSA than Baxter HSA. This implies that Baxter HSA is more stable to Pronase® degradation than fat free DBax HSA. The stability towards enzymatic degradation of Bax HSA is similar to that of HSAfa whilst fat free DBax HSA is similar to that of HSAff.

Delipidised (Fat Free) Baxter HSA (DBax HSA) with RH01 (1:2) in the Presence of Pronase® 1/100 w/w.

The lipopeptide RH01 stabilises fat free DBax HSA like HSAff and in doing so the lipopeptide itself is stabilised by DBax HSA resulting in mutual stabilisation.

The discussion of the following examples 15-27 is given after example 27.

### Example 15

### Antimicrobial Activity of Fat-Free Albumin with Lipopeptide

RH01 (3 mg) was added to human serum albumin essentially globulin-free and fatty acid-free (HSAff) (25 mg) purchased from Sigma (lot. 32H9300). Sterile phosphate buffered saline (PBS) (1.5 ml) was added to the mixture under aseptic condition. Solution, was assayed for antimicrobial activities using S. aureus and E. coli as below.

Bacterial Strain Assays

Staphylococcus aureus NCTC Oxford and Escherichia coli 0111-NCTC 8007 strains were obtained from the National Collection of Type Cultures, Colindale, UK MIC for each sample was determined in 96 well plates. The above sample in PBS was serially diluted in microtitre wells with media, RPMI-1640 to give concentrations from 2 mg/ml to 0.00375 mg/ml or from 1 mg/ml to 0.00375 mg/ml or from 0.5 mg/ml to 0.00375 mg/ml of RH01 in a final volume of 100 μl. Bacteria were incubated at 37° C. overnight in standard media to give approximately 10⁸ bacteria/ml and 10 μl of this was added to each well. The plates were incubated at 37° C. overnight, and bacterial growth determined by formation of a pellet. The MIC for each sample was determined in triplicate as the concentration required to completely inhibit bacterial growth.

### Example 16

### Antimicrobial Activity of Albumin with Fatty Acid with Lipopeptide RH01 6 Molar Per Molar Albumin

RH01 (3 mg) was added to human serum albumin essentially globulin-free (HSAfa) (25 mg) purchased from SigmaAldrich Company Ltd (lot. 105H9300). Sterile phosphate buffered saline (1.5 ml) was added to the mixture under aseptic condition. Solution assayed for antimicrobial activities using S. aureus and E. coli.

Bacterial strains used were as in Example 15.

### Example 17

### Antimicrobial Activity of Lipopeptide RH01 Alone

RH01 (3 mg) was placed in a glass vial. Sterile phosphate buffered saline (1.5 ml) was added to the mixture under aseptic condition. Solution assayed for antimicrobial activities using S. aureus and E. coli.

Bacterial strains used were as in Example 15.

### Example 18

### Antimicrobial Activity of Albumin Fatty Acid Free with Lipopeptide RH01 in the Presence of Palmitic Acid 0.7, 0.8, 1 Molar Per Molar Albumin

Sodium palmitate, PA (0.63 mg), was dissolved in ethanol (439 μl) and sonicated for 5 mins. The fatty acid is normally allowed to equilibrate with albumin by incubation for one and a half hours in the albumin-fatty acid mixture.

HSAff (5.064 mg) was dissolved in PBS (560 μl) and PA in ethanol solution (15 μl) was added to HSAff and gently stirred and left for 1.5 h. HSAff+PA solution (509 μl) was added to a glass vial containing RHOL (0.0491 mg) and gently mixed and left at room temperature for 30 mins giving molar ratio of the mixture RH01:HSAff:PA (6:1:1). Further solutions were prepared accordingly to give molar ratio of the mixture RH01:HSAff:PA (6:1:0.7) and (6:1:0.8).

Solution assayed for antimicrobial activities using S. aureus. Bacterial strains used were as in Example 15.

### Example 19

### Antimicrobial Activity of Albumin Fatty Acid Free with Lipopeptide RH01 in the Presence of Palmitic Acid 2 Molar Per Molar Albumin

Sodium palmitate, PA (0.63 mg), was dissolved in ethanol (439 μl) and sonicated for 5 mins.

HSAff (5.298 mg) was dissolved in PBS (571 μl) and PA in ethanol solution (31 μl) was added to HSAff and gently stirred and left for 1.5 h. HSAff+PA solution (527 μl) was added to a glass vial containing RH01 (0.527 mg) and gently mixed and left at room temperature for 30 mins giving molar ratio of the mixture RH01:HSAff:PA (6:1:2). Solution assayed for antimicrobial activities using S. aureus.

Bacterial strains used were as in Example 15.

### Example 20

### Antimicrobial Activity of Albumin Fatty Acid Free with Lipopeptide RH01 in the Presence of Palmitic Acid 4 Molar Per Molar Albumin

Sodium palmitate, PA (0.63 mg), was dissolved in ethanol (439 μl) and sonicated for 5 mins.

HSAff (5.695 mg) was dissolved in PBS (578 μl) and PA in ethanol solution (65 μl) was added to HSAff and gently stirred and left for 1.5 h. HSAff+PA solution (591 μl) was added to a glass vial containing RH01 (0.591 mg) and gently mixed and left at room temperature for 30 mins giving molar ratio of the mixture RH01:HSAff:PA (6:1:4). Solution assayed for antimicrobial activities using S. aureus.

Bacterial strains used were as in Example 15.

### Example 21

### Antimicrobial Activity of Lipopeptide in the Presence of Palmitic Acid 1 Molar Per Molar Lipopeptide

Sodium palmitate, PA (0.848 mg), was dissolved in ethanol (586 μl) and sonicated for 5 mins.

RH01 (2.171 mg) was dissolved in PBS (2.171 ml) and PA in ethanol solution (15 μl) was added to RH01 (600 μl) and gently stirred and left for 1.5 h. giving a molar ratio of the mixture RH01:PA (1:1). Solution assayed for antimicrobial activities using S. aureus.

Bacterial strains used were as in Example 15.

### Example 22

### Antimicrobial Activity of Palmitic Acid Alone

Sodium palmitate, PA (0.63 mg), was dissolved in ethanol (439 μl) and sonicated for 5 mins.

PA in ethanol solution (60 μl) was added to PBS (600 μl) and gently stirred and left for 2 h at 37° C. Another sample was prepared and left standing at room temperature for 2 h. Solution assayed for antimicrobial activities using S. aureus.

Bacterial strains used were as in Example 15.

### Example 23

### Antimicrobial Activity of Caprylic Acid Alone

Sodium caprylate, CA (0.344 mg), was dissolved in distilled water (5.73 ml).

CA (60 μl) was added to PBS (600 μl) and gently stirred and left for 2 h at 37° C. Another sample was prepared and left standing at room temperature for 2 h. Solution assayed for antimicrobial activities using S. aureus.

Bacterial strains used were as in Example 15.

### Example 24

### Antimicrobial Activity of Lipopeptide RH01 with Caprylic Acid 1 Molar Per Molar Lipopeptide

RH01 (3 mg) was added to sodium caprylate (0.107 mg). Sterile phosphate buffered saline (PBS) (1.5 ml) was added to the mixture under aseptic condition giving molar ratio of RH01:CA (1:1). Solution was assayed for antimicrobial activities using S. aureus and E coli.

Bacterial strains used were as in Example 15.

### Example 25

### Antimicrobial Activity of Albumin Fatty Acid Free with Lipopeptide RH01 in the Presence of Caprylic Acid 6 Molar Per Molar Albumin

RH01 (3 mg) was added to sodium caprylate (0.1 mg) and HSAff (25.3 mg). Sterile phosphate buffered saline (PBS) (1.5 ml) was added to the mixture under aseptic condition giving molar ratio of RH01:HSAff-CA (6:1-6). Solution was assayed for antimicrobial activities using S. aureus and E. coli.

Bacterial strains used were as in Example 15.

### Example 26

### Antimicrobial Activity of Commercial Baxter HSA (Bax HSA) with Lipopeptide RH01 6 Molar Per Molar Albumin

RH01 (0.338 mg) was dissolved in sterile phosphate buffered saline (610 μl). Bax HSA 4.5% (66 μl) was added to RH01 solution to form a solution of RH01 0.5 mg/ml giving the molar ratio of RH01:Bax HSA (6:1). Solution assayed for antimicrobial activities using E. coli.

Bacterial strains used were as in Example 15.

### Example 27

### Antimicrobial Activity of Fat Free Delipidised Baxter HSA (DBax HSA) with Lipopeptide RH01 6 Molar Per Molar Albumin

RH01 (0.382 mg) was dissolved in sterile phosphate buffered saline (611 μl). DBax HSA 2.21% (153 μl) was added to RH01 solution to form a solution of RH01 0.5 mg/ml giving the molar ratio of RH01:DBax HSA (6:1). Solution assayed for antimicrobial activities using E. coli.

Bacterial strains used were as in Example 15.

Results

Minimum Inhibitory Concentrations (μM) found are illustrated in the table 1 below.

TABLE 1
Minimum inhibitory concentrations (MIC) of
lipopeptide RH01 alone and in the presence of
albumins (HSAff and HSAfa).

Minimum Inhibitory Concentration (μM)

| Samples | E. coli | S. aureus |
|---|---|---|
| RH01 + HSAff | 24 | 12 |
| RH01 + HSAfa | 48 | 24 |
| RH01 | 48 | 24 |

It was shown that the presence of HSAff in the mixture containing antimicrobial RH01 resulted in a lower MIC against E. coli and S. aureus compared to that of the peptide alone and the mixture with HSAfa.

The results show that the HSAff enhances the antimicrobial activity of RH01. With this lipopeptide, the effect is twice as potent as compared to the lipopeptide alone or with HSAfa. This can be seen as a reduction in the dosage by half. The results also demonstrate that exogenous standard albumin with fatty acid (HSAfa) has no beneficial effect on the potency when used in conjunction with the lipopeptide RH01.

TABLE 2
Minimum inhibitory concentrations (MIC) of
RH01 and RH01 with albumins against
S. aureus in the presence of fatty acids

MIC (μM) Peptide
incubation time in mixture

| | 2 h 37° C. | 30 min 25° C. |
|---|---|---|
| Samples | S. aureus | S. aureus |
| RH01 + HSAff | 12 | 6 |
| RH01 + HSAfa | 24 | 12 |
| RH01 | 24 | 12 |
| RH01 + CA | 24 | n/a |
| RH01 + HSAff + CA (6:1:1) | 48 | n/a |
| RH01 + PA (6:1) | n/a | 12 |
| RH01 + HSAff + PA (6:1:0.7) | n/a | 12 |
| RH01 + HSAff + PA (6:1:0.8) | n/a | 12 |
| RH01 + HSAff + PA (6:1:1) | n/a | 12 |
| RH01 + HSAff + PA (6:1:2) | n/a | 24 |
| RH01 + HSAff + PA (6:1:4) | n/a | 24 |
| CA | >800 | >800 |
| PA | >800 | >800 |

In table 2, the results show that on addition to fatty acids (palmitic acid) to a mixture of lipopeptide and HSAff, the MIC are increased to fourfold for the higher concentration of palmitate consistent with a decreased antimicrobial activity of RH01. It is important to note that the effect of 0.7, 0.8 and 1 mole of palmitic acid per mole of HSAff in HSAff-RH01 mixture is similar to that of HSAfa-RH01. This means that the best results are achieved with albumin containing less than 0.7 mole of fatty acid per mole of albumin.

TABLE 3
Minimum inhibitory concentrations (MIC)
of lipopeptide RH01 in the presence of albumins (Baxter
HSA (Bax HSA) and delipidised Baxter HSA (DBax HSA)).

| | Minimum Inhibitory Concentration (μM) |
|---|---|
| Samples | E. coli |
| RH01 + DBax HSA | 24 |
| RH01 + Bax HSA | 48 |

In table 3, the results show that DBax HSA in the mixture containing antimicrobial RH01 results in, a lower MIC against E. coli compared to the mixture with Bax HSA. The commercial Baxter HSA solution containing sodium caprylate behaves similarly to HSAfa containing fatty acids whilst the delipidised Baxter HSA behaves like fatty acid free HSAff.

This is consistent with the results obtained for HSAff and HSAfa shown in table 1.

With this lipopeptide peptide model, the peptide was bound to the fatty acid binding site of HSAff and DBax HSA, thus resisting hydrolysis by bacterial enzymes and able to exert its antimicrobial activity at a lower concentration. When the peptide is used in conjunction with HSAfa and Baxter HSA, the MIC is the same as that of the peptide alone, HSAfa confers no further stability to the peptide since most of the peptide is in the unbound state, hence making it susceptible to bacterial enzymatic degradation. When the fatty acid palmitate was added, the samples antimicrobial activity was reduced indicating the displacement of bound lipopeptide thus making it more susceptible to bacterial enzymatic hydrolysis. This is consistent with the mutual stabilisation effect of lipopeptides on HSA as in previous examples.

This invention also relates to the use of peptides of the kind described above, and additional peptides specified hereinafter, as antimicrobials.

Infection and autoimmunity are the most common and rampant cause of diseases. Current available therapies and drugs are showing signs of failure in treatment efficacy. Microbes are increasingly developing defensive mechanisms against known drugs via mutations. Already there are signs of emergence of superbugs which are immune to most known antibiotics available. The need for a new class of drugs to counteract this problem is of paramount importance for continued general well being of mankind. A class of drugs, antimicrobial peptides, has now emerged which have as yet not been fully exploited.

We have also discovered that these peptides have antimicrobial properties and act on bacterial membranes. This antimicrobial action is less susceptible to the development of microbe resistance and mutation, thus ensuring a better efficacy of this new class of antimicrobial agents. The peptides are specified hereinafter by standard single letter symbols for their component amino acids.

Representative peptides having antimicrobial activities having or containing the sequence:

FARKGALRQ(SEQ.ID NO:1)

including:

| | |
|---|---|
| KFAPKGALRQKNK | (SEQ. ID NO:2) |
| KFARKGALRKKNK | (SEQ. ID NO:3) |
| KFKRKGALRQKNK | (SEQ. ID NO:4) |

These may be acylated or derivatised peptides as in the following:

RH01, Myristoylated-FARKGALRQ,

RH02, (Pm)ICFARKGALRQKNK(Pm)-amide,

RH03, (Pm)KFARKGALRK(Pm)KNK(Pm)-amide,

RH04, KFKRKGALRQKNIC-amide, where Pm is Palmitoyl moiety.

The term antimicrobial means that the peptides of the present invention inhibit, prevent or destroy the growth or proliferation of microbes such as bacteria, fungi, viruses or the like. These peptides may be used in human and animal treatments and in agriculture.

Minimum Inhibitory Concentration (MIC):

In the present invention, the minimum inhibitory concentration of peptides RH01, RH02, RH03 and RH04 were ascertained with gram negative bacteria, Escherichia coli and a gram positive bacteria, Staphylococcus aureus.

### Example 28

### Synthesis of RH01

The peptide was synthesised using standard procedures, on a solid phase peptide synthesiser (Applied Biosystems 430A) using standard tert-butyloxycarbonyl, BOC/trifluroacetic acid, TFA chemistry. A chloromethylated resin (Fluka) (0.5 mmol) was used. L-amino acids (2 mmol) were used in the synthesis with amino acid protecting groups as follows: Arginine, tosyl; Lysine, chlorobenzyloxycarbonyl. The resin (0.73 g) containing the terminal N-Boc Phenylanine residue was then deprotected with trifluoroacetic acid in dichloromethane (50%) (30 ml) and stirred for 1 hour. The resin was filtered and washed three times with dichloromethane (30 ml) each time. The resin was then washed three times with N,N-diisopropylethanolamine, DIEA (30 ml), and finally three times with dicholoromethane (30 ml). Myristic acid (0.285 g), (benzotriazol-1-yloxy)tris(dimethyl-amino)phosphonium hexafluorophosphonate, BOP (1.1 g), 1-hydroxybenzotriazole, HOBt (0.333 g), DIEA (1.375 g, 1.85 ml), and N-methylpyrrolidone, NMP (30 ml), were added to the washed resin and the mixture was stirred for 2 hours. The resin was filtered and washed with dichloromethane (30 ml). The crude peptide was liberated from the resin by anhydrous hydrogen fluoride, HF (10 ml), cleavage.

The peptide was purified using a preparative C18 RP-Nucleosil column. The HPLC analytical conditions used were a solvent gradient 0-100% of 0.05% TFA, and 50% acetonitrile in water over 30 minutes. The peptide detection was monitored by absorbance at 215 nm.

The primary characterisation of the peptide was performed using time-of-flight plasma desorption mass spectrometry.

### Example 29

### Synthesis of RH02

The peptide was synthesised by solid phase synthesis as in example 1 using 4-methyl benzyhdrylamine resin, Boc-Fmoc-lysine and palmitic acid. The peptide resin which contain Fmoc-lysine was then place in a reaction vessel and piperidine 20% in dimethyformamide, DMF, was added to the vessel. The mixture was allowed to react for 20 minutes. The resin was then filtered and washed with 3 times with DMF (30 ml) and 3 times with DCM (30 ml). Palmitic acid (0.128 g), BOP (0.354 g), HOBt (0.108 g), DIEA (0.44 g, 0.6 ml), and N-methylpyrrolidone, NMP (5 ml), were added to the washed resin and the mixture was stirred for 2 hours. The resin was filtered and washed with dichloromethane (30 ml). The crude peptide was liberated from the resin by anhydrous hydrogen fluoride, HF (10 ml), cleavage.

### Example 30

### Synthesis of RH03

The peptide was synthesised by solid phase as in example 1 and 2.

### Example 31

### Synthesis of RH04

The peptide was synthesised as in example 1 using 4-methyl benzyhdrylamine resin.

### Example 32

### Antimicrobial Activity of RH01

RH01 (3 mg) was dissolved in sterile phosphate buffered saline (PBS) (1.5 ml) under aseptic condition and was then left for 30 mins at 37° C. or at 25° C. Solution was assayed for antimicrobial activities using S. aureus and E. coli as shown below.

Bacterial Strains

Staphylococcus aureus NCTC Oxford and Escherichia coli 0111-NCTC 8007 strains were obtained from the National Collection of Type Cultures, Colindale, UK. The MIC for each sample was determined in 96 well plates. The RH01 in PBS (see above) was serially diluted in microtitre wells with media (RPMI-1640) to give final concentrations of 2 mg/ml to 0.00375 mg/ml of RH01 in a final volume of 100 μl. Bacteria were incubated at 37° C. overnight in standard media to give approximately 10⁸ bacteria/ml and 10 μl of this was added to each well. The plates were incubated at 37° C. overnight, and bacterial growth determined by formation of a pellet. The MIC for each sample was determined (in triplicate) as the concentration required to completely inhibit bacterial growth.

### Example 33

### Antimicrobial Activity of RH02

RH02 (1.368 mg) was dissolved in sterile phosphate buffered saline (PBS) (684 μl) under aseptic condition and was then left for 30 mins at 37° C. Solution was assayed for antimicrobial activities using S. aureus and E. coli as shown in example 5.

### Example 34

### Antimicrobial Activity of RH03

RH03 (1.452 mg) was dissolved in sterile phosphate buffered saline (PBS) (726 μl) under aseptic condition and was then left for 30 mins at 37° C. Solution was assayed for antimicrobial activities using S. aureus and E. coli as shown in example 5.

### Example 35

### Antimicrobial Activity of RH04

RH04 (1.716 mg) was dissolved in sterile phosphate buffered saline (PBS) (858 μl) under aseptic condition and was then left for 30 mins at 37° C. Solution was assayed for antimicrobial activities using S. aureus and E. coli as shown in example 5.

The results (table 4) show the peptide has a potent antimicrobial activity.

TABLE 4
Minimum inhibitory concentration of RH01 and its
derivatives required to completely inhibit bacterial growth

Minimum Inhibitory Concentration (μM)

| | |
|---|---|
| 25° C. | 37° C. |

| Sample | S. aureus | E. coli | S. aureus |
|---|---|---|---|
| RH01 | 12 | 48 | 24 |
| RH02 | — | 62 | 3.7 |
| RH03 | — | 441 | 55 |
| RH04 | — | 620 | 145 |

Additional useful peptides are those having or containing the sequences:

| | |
|---|---|
| DVANRFARKGALRQKNVLIEVK, | seq ID 5. |
| ESTVRFARKGALRQKNVHIEVK, | seq ID 6. |

The peptides can be acylated on the N-terminus and or C-terminus and/or suitable amino acid side chain residues in the peptides. Furthermore, the peptides can be esterified on the C-terminus and/or suitable amino acid side chain residues of peptides.

These peptides can be administered by oral, inhalational (oral and nasal), transdermal, parenteral and other mucosal routes (such as vaginal, rectal, opthalmic and buccal mucosa), at dosages in the range of 1 mg-1 g, and preferably in the range 50 mg-1 g.

1. An exogenous pharmaceutical preparation comprising a bioactive substance covalently attached to a lipidic tagging group, the tagged substance being non-covalently bound to an albumin initially containing a proportion of lipidic groups of no more than 0.5 mole of fatty acid per mole of albumin.

2. An exogenous pharmaceutical preparation according to claim 1, in which the albumin is initially substantially fat-free.

3. An exogenous pharmaceutical preparation according to claims or 2, for human use.

4. An exogenous pharmaceutical preparation according to claims or 2, for veterinary use.

5. An exogenous pharmaceutical preparation according to any of the preceding claims, in which the albumin is a serum albumin.

6. An exogenous pharmaceutical preparation according to any of the preceding claims, in which the albumin is a serum albumin.

7. An exogenous pharmaceutical preparation according to any of the preceding claims, in which the albumin is a recombinant serum albumin.

8. An exogenous pharmaceutical preparation according to any of the preceding claims, in which the bioactive substance is a peptide or protein.

9. An exogenous pharmaceutical preparation according to claim 8, in which the bioactive substance is a peptide selected from the group consisting of:

| | |
|---|---|
| FARKGALRQ | (SEQ. ID. NO:1) |
| KFARKGALRQKNK | (SEQ. ID. NO:2) |
| KFKRKGALRQKNK | (SEQ. ID. NO:3) |

10. An exogenous pharmaceutical preparation according to any of the preceding claims, in which the bioactive substance is a vaccine antigen.

11. An exogenous pharmaceutical preparation according to any of the preceding claims, in which the lipidic tagging group is a C₄-C₁₆ single chain fatty acid.

12. The use of an albumin initially containing a proportion of lipidic groups of no more than 0.5 mole of fatty acid per mole of albumin for the preparation of an exogenous composition comprising a bioactive substance covalently attached to a lipidic tagging group, in which the the tagged substance is non-covalently attached to the albumin

13. The use according to claim 13, in which the albumin used is substantially free of fatty acid groups.

14. A method of conferring mutual stability on a lipopeptide or lipoprotein and an albumin carrier therefor, which comprises incorporating the lipopeptide or lipoprotein in an exogenous composition with an albumin initially containing a proportion of fatty acid groups of no more than 0.7 mole of fatty acid per mole of albumin.

15. A method of determining the fatty acid content of an albumin by displacement of a marker substance and circular dichroism spectroscopy.

16. The antimicrobial use of a peptide having or containing the sequence FARKLGALPQ (SEQ. ID No: 1)

17. The use of a peptide according to claim 16 in which the peptide has or contains a sequence selected from:

| | |
|---|---|
| KFARKGALRQKNK | (SEQ. ID. NO:2) |
| KFARKGALRKKNK | (SEQ. ID. NO:3) |
| KFKRKGALRQKNK | (SEQ. ID. NO:4) |
| DVANRFARKGALRQKNVHEVK | (SEQ. ID. NO:5) |
| ESTVRFARKGALRQKNVHEVK | (SEQ. ID. NO:6) |

18. The peptides according to claim 16 or 17 which are acylated or derivatised on the N-terminus and/or C-terminus and/or suitable amino acid side chain residues.

19. The peptides according to claim 16 or 17 which are esterified or derivatised on the C-terminus and or suitable amino acid side chain residues.

20. The use of a peptide according to any of claims 16-19, in the preparation of a medicament for human use.

21. The use of a peptide according to any of claims 16-19, in the preparation of a medicament for veterinary use.

22. The use of a peptide according to any of claims 16-19, for agricultural use.

23. A composition for pharmaceutical, veterinary, or agricultural use containing a peptide according to any of claims 16-19.

24. A method of inhibiting, preventing, or destroying the growth or proliferation of microbes, using peptides as defined in any of the preceding claims.
